(19) United States
(12) Patent Application Publication
(10) Pub. No.: US 2002/0084221 A1
(43) Pub. Date: Jul. 4, 2002

(54) Method for precipitating red blood cells

(76) Inventors: Wesley H. Verkaart, Duxbury, MA (US); James R. Ellsworth, Marshfield, MA (US)

Correspondence Address: Clark & Brody, Suite 600, 1750 K Street, NW, Washington, DC, 20006, US

(21) Appl. No.: 10/001,966

(22) Filed: Dec. 5, 2001

Related U.S. Application Data
(60) Provisional application No. 60/251,047, filed on Dec. 5, 2000.

(51) Int. Cl. A01N 1/02; A61K 31/727; B01D 11/00

(52) U.S. Cl. 210/647; 435/2; 514/56; 514/60

(57) ABSTRACT

A method for washing collected blood uses an inert anticoagulant during collection of the blood. The inert anticoagulant does not interfere with agglomeration of the red blood cells so that gravity sedimentation separation is facilitated. The preferred inert anticoagulant is CPD.

### CROSS REFERENCE TO RELATED APPLICATION

This application claims the priority of U.S. Provisional Patent Application serial No. 60/251,047, which was filed on Dec. 5, 2001.

### TECHNICAL FIELD

This invention relates to methods for collecting, washing, and returning blood to a patient. In particular the invention relates to methods for efficiently separating red blood cells from washing or other liquids in preparation for transfusing the red blood cells to the patient.

### BACKGROUND OF THE INVENTION

Methods for collecting blood and processing it for transfusion back to a patient are known. For example, blood shed during surgery is often collected for the purpose of re-infusing the blood during surgery. The shed blood that has been collected may be washed before it is re-infused. This may typically include mixing the collected blood with a wash solution and then separating the red blood cells from the solution, which retains the unwanted substances. In some known systems, the red blood cells are separated by a gravity process in which the red blood cells settle to the bottom of a container because they are more dense than the wash solution and other components in the mixture. Centrifugal devices are also known for use in washing cells, the separation between the liquid and the red blood cells being accomplished by application of centrifugal forces.

Known apparatus for separating red blood cells by sedimentation include those shown in U.S. Pat. No. 5,282,982 and published PCT application WO 99/44711. In general, these structures operate by providing a shallow container for receiving the mixture of red blood cells and washing solution. The container is tilted so that the red blood cells, which settle out of the solution by gravity due to density differences, flow down bottom surfaces of the container to a collection funnel for discharge to the re-infusion device.

The methods of the described prior systems also include the step of collecting the blood into a container having ACD-A as the anticoagulant therein and the step of adding a reagent that facilitates aggregation of the red blood cells. The preferred reagent is hetastarch, which allows remote red blood cells to be electrically attracted to each other whereby they aggregate and form a clump in a stacked roll configuration known as a rouleau. It is believed that the hetastarch molecules promote formation of a rouleau by forming an electrical bridge between the remote red blood cells. The rouleau exhibits smaller hydrodynamic drag in the solution and, thus, is expected sink to the bottom of the container more quickly.

The problem faced by applicants is that the above-described process is not reliable in practice. That is, the rouleau in that process often failed to form as required or to settle out of the plasma-hetastarch solution in a reasonable amount of time to provide a solution with a hematocrit adequate for re-infusion to the patient.

### SUMMARY OF THE INVENTION

Applicants have discovered that a serious obstacle in the prior process is that the anticoagulant employed has a major effect on the viability of the process. Thus, applicants have discovered that a process wherein the anticoagulant mixed with the shed blood that is not the usual ACD-A provides remarkably improved results. That is, in accordance with the invention, a cell salvage process wherein shed blood is combined with only inert anticoagulants and then mixed with a washing solution having a reagent, such as hetastarch, successfully separates red blood cells in a gravity sedimentation method.

As used herein “inert” or “inert anticoagulant” means an anticoagulant that prevents coagulation but does not affect the ability of red blood cells to rouleau effectively for separation by sedimentation. One such inert anticoagulant discovered by applicants is citrate phosphate dextrose (CPD). Heparin is another inert coagulant, which does not hinder the formation of the rouleau. Heparin, however, is less preferred for use in the method of the invention because the blood is usually saved for the purpose of returning it to a patient. Heparin may present an undesirable effect in the patient, and it may not be appropriate to return such blood to the patient. Thus, the preferred anticoagulant is CPD because it does not interfere with sedimentation of red blood cells and because the patient can metabolize it easily.

Applicant believes that the prior art method was not useful because the anticoagulant used in the process, ACD-A, has an adverse effect on the red blood cells. In particular, it is now believed that the absorption of ACD-A by the red blood cells has a physical effect on the cells that prevents them from forming the desired rouleau and settling out under gravitational forces as desired. One theory developed by applicants is that the absorption of ACD-A changes the shape of the red blood cells, swelling them and hindering their ability to form the rouleau. Other reasons for interference by the anticoagulant may exist.

Inert anticoagulants other than CPD and heparin might be discovered or developed. Further, it may be possible to treat ACD-A, physically or chemically, such that it becomes inert for this purpose.

Further, washing solutions having reagents other that hetastarch may be used. It is known that red blood cells are electrically attracted to each other, and hetastarch appears to assist in the formation of the rouleau by providing an electrical bridge between remote red blood cells. Other reagents may be found to promote the formation of a rouleau, including other starch reagents such as pentastarch. Thus the invention contemplates the use of other washing solutions having other reagents.

### DETAILED DESCRIPTION OF THE INVENTION

In a preferred embodiment, blood is collected into a container by a vacuum system that applies controlled vacuum of small pressure differential to reduce damage to the red blood cells during collection. This may be accomplished with the system disclosed in WO 99/44711 or other systems known in the art. The collected blood is mixed with CPD as the inert anticoagulant as it is collected. CPD is added to blood at a ratio of CPD-to-blood adequate to prevent coagulation of the blood during the procedure. The ratio is up to one part CPD to fifteen parts blood, preferably in the range of from 1:5 to 1:15 and more preferably one part CPD to ten parts collected blood.

Then, the collected blood is mixed with a washing solution having a reagent (preferably saline with 6% hetastarch) and placed in a sedimentation chamber for processing. Preferably the hetastarch is added at a rate of 8 parts hetastarch to 5 parts of a blood/anticoagulant mixture, but a wide range of ratios may be found useful to remove unwanted materials from the blood.

The solution is then left undisturbed for a period of time to allow most, if not all, of the red blood cells to rouleau and settle to the bottom of the container. The period of time varies, but it has been found that about twenty minutes is sufficient. The red blood cells are then removed from the container conventionally and re-infused into the patient.

Clinical trials implementing the above technique with the apparatus disclosed in WO 99/44711 have shown that the red blood cells are separated from the washing solution in about twenty minutes, with the hematocrit of the saved cells ranging from 30-64 Hct.

Modifications within the scope of the appended claims will be apparent to those of skill in the art.

We claim:

1. In a method for separating by sedimentation red blood cells from a solution including blood and an anticoagulant, the improvement wherein said anticoagulant consists essentially of an inert anticoagulant.

2. The method of claim 1 wherein said inert anticoagulant is CPD.

3. The method of claim 1 wherein said CPD is mixed with said blood at a ratio of one part CPD and up to fifteen parts blood.

4. The method of claim 1 wherein said inert anticoagulant is heparin.

5. In a method for separating by sedimentation red blood cells from a solution including blood and an anticoagulant, the improvement wherein said anticoagulant consists essentially of an anticoagulant selected from the group consisting of CPD and heparin.

6. A method for washing blood comprising the steps of providing said blood with an inert anticoagulant, mixing said blood with a washing solution, and allowing red blood cells to separate from said washing solution by sedimentation.

7. A method according to claim 6 wherein said inert anticoagulant is CPD.

8. A method according to claim 7 wherein said washing solution includes a starch solution.

9. A method according to claim 7 wherein said washing solution includes hetastarch.

10. A method according to claim 7 wherein said washing solution is 6% hetastarch.

11. A method according to claim 6 wherein said inert anticoagulant is heparin.

12. A solution comprising blood, anti-coagulant, and a washing solution, wherein said anticoagulant is selected from the group consisting of CPD and heparin.

13. A solution according to claim 12 wherein said washing solution comprises starch.

14. A solution according to claim 13 wherein said starch is hetastarch.

15. A solution consisting essentially of blood, an inert anti-coagulant, and a washing solution.

16. A solution according to claim 15 wherein said inert anticoagulant is CPD.

17. A solution according to claim 15 wherein said inert anticoagulant is heparin.

18. A solution according to claim 15 wherein said blood is a sediment layer of blood.

19. A solution according to claim 18 wherein said sediment layer contains a majority of the red blood cells of said blood.

20. A solution according to claim 19 wherein said sediment layer is about 30-64 Hematocrit.
